(19) United States
(12) Patent Application Publication
(10) Pub. No.: US 2022/0400998 A1
(43) Pub. Date: Dec. 22, 2022

(54) TOILET DEVICE FOR NON-CONTACT MEASUREMENT OF MICTURITION PARAMETERS

(71) Applicant: MEDIPEE GMBH, Moers (DE)

(72) Inventors: Thomas PROKOPP, Bonn (DE); Paul BANDI, Kamp-Lintfort (DE); Marcel MORCINCZYK, Rheinberg (DE); Jan HAAN, Moers (DE); Frank WILLEMS, Moers (DE)

(21) Appl. No.: 17/779,715

(22) PCT Filed: Nov. 26, 2020
(86) PCT No.: PCT/EP2020/083486

§ 371 Date: May 25, 2022

(30) Foreign Application Priority Data
Nov. 26, 2019 (DE) 10 2019 132 005.4

(51) Int. Cl. A61B 5/20; A61B 5/11; A61B 5/00; E03D 11/00

(52) U.S. Cl. CPC A61B 5/207; A61B 5/1114; A61B 5/7264; E03D 11/00; A61B 2562/0271; A61B 2562/0219; A61B 2562/0257; A61B 2562/12; A61B 2562/164; A61B 2562/046; A61B 2560/0242; A61B 5/0002

(57) ABSTRACT

The invention relates to a toilet device for the measurement of micturition parameters, the toilet device comprising a housing that has a housing opening for receiving urine and a capacitive sensor for the time-dependent, contactless measurement of micturition parameters. The invention also relates to a method for the contactless measurement of micturition parameters in a toilet device, said method being carried out more particularly using the toilet device according to the invention.

The invention relates to a toilet device for measuring micturition parameters, wherein the toilet device comprises a housing with a housing opening for receiving urine and a capacitive sensor for time-dependent contactless measurement of micturition parameters. The invention also relates to a method for contactless measurement of micturition parameters in a toilet device, which is carried out in particular using the toilet device according to the invention.

The excretion of urine, which is also referred to as urination, bladder emptying or micturition, is physiologically very important for the body for several reasons. First, the body's water balance is regulated in this way. In addition, substances are excreted with the urine that accumulate during metabolism and are no longer needed by the body. This also includes toxic substances that have been ingested with food, or medications. Examination of the urine can reveal indications of diseases of the kidney and urinary system, but can also detect metabolic diseases such as diabetes or diseases of the liver. In addition to the actual content analysis of the urine, an analysis of urine flow parameters can also be performed. Uroflowmetry is used for this purpose. Uroflowmetry is a diagnostic procedure that measures urine flow during micturition. It is used to objectively determine bladder emptying disorders and is one of the basic examinations in urology. By measuring the urine stream volume per time interval (e.g. in ml/s), a flow curve is created in which the amount of urine passed is plotted against time. This flow curve shows a typical course in certain diseases. The disadvantage here is that this measurement must be carried out in a clinic or practice under supervision and a special examination device is required for this. In addition, it is a prerequisite for a properly performed measurement that the bladder of the test person is sufficiently filled. The subject must wait with micturition (emptying the bladder) until he or she feels a strong sensation of pressure. The result of uroflowmetry is only meaningful if the volume of urine exceeds 150 ml. In addition, the collection of urine over a defined period of time (preferably 24 h) may be necessary to check the functional efficiency of the organ, especially in the case of kidney disease. Frequently, the urine volume delivered is checked in the context of transplantations.

It is the task of the invention to provide an improved method and a toilet device for measuring micturition parameters.

According to the invention, this task is solved by a toilet device having the features of independent claim 1. Advantageous further embodiments of the toilet device result from dependent claims 2 to 10. In a further aspect, this task is solved by a method for measuring micturition parameters having the features of claim 11. Advantageous further embodiments of the method result from dependent claims 12 to 14.

### SUMMARY OF THE INVENTION

In a first aspect, the invention provides a toilet device for measuring micturition parameters, wherein said toilet device comprises a housing having a housing opening for receiving urine and a capacitive sensor for time-dependent measurement of micturition parameters, and wherein the capacitive sensor is a capacitive proximity sensor.

The method according to the invention combines several decisive advantages over methods known from the prior art.

Due to the non-contact measurement by means of the capacitive proximity sensor, the sensor is not contaminated by urine or feces. Therefore, no time-consuming and costly cleaning of the sensor is necessary, but the sensor is always ready for operation without external intervention.

This means that automatic digitalized measurement of micturition parameters can now be carried out in the home environment for the first time. The test person or patient does not have to go to a practice or clinic first, but the measurement can be integrated into his everyday life in a simple and for him undisturbing way.

The non-contact measurement also reduces the risk of analysis results being falsified. In addition, the capacitive sensor can be retrofitted in a simple manner to or on an existing toilet facility, thus allowing an existing toilet facility to be retrofitted.

Furthermore, the measuring device according to the invention can be combined with all housing-supporting toilet devices and can thus be used in a broad manner for the most diverse types of toilets.

In its simplest (i.e. unshielded) variant, the capacitive sensor permits measurement independent of the space and thus, in addition to measuring the micturition parameters inside the housing, it can also detect processes outside the toilet device, such as the approach and sitting down of the toilet user.

The simplifications of the device proposed here not only result in a higher reliability of the device, but also reduce the weight of the device due to the associated reduction of components, as well as an extremely efficient operation of the device. In addition, they allow immediate measurement almost without delay.

In the sense of the invention, the term “toilet device” covers any sanitary device for receiving bodily excreta (in particular feces and urine).

According to the invention, the term “housing” is understood to mean a vessel as a component of a toilet device which can receive urine. The vessel preferably has a watertight (and thus urine-tight) vessel wall, which is particularly preferably designed as a rigid and stiff wall, so that the housing can serve, for example, as a seat or seat base for the toilet user. The housing may have other openings in addition to the housing opening provided for urine collection, such as for the disposal of excrement or the supply of flushing fluid.

According to the invention, the term “housing opening” is defined as the opening of a housing of a toilet device through which the urine is received. According to the invention, this term covers not only the opening as such, but also a means which is mounted or arranged on at least part of the edge of the opening and thus forms part of the opening. Thus, a toilet seat as a structure resting on the rim is also covered by the term housing opening. According to the invention, the capacitive sensor is a capacitive proximity sensor which reacts without contact—i.e. without direct contact—to the approach of liquids, i. e. an electrical sensor. It utilizes the changing electrical capacitance of a measuring electrode to the environment or a reference electrode.

According to the invention, the capacitive sensor is set up in such a way that the capacitance change is caused by introducing an electrically conductive material, such as urine or a dielectric, into the immediate vicinity of the capacitive sensor. According to the invention, the “immediate environment” means an environment at a distance of less than 20 cm, preferably less than 10 cm and further preferably less than 5 cm. This can be achieved on the one hand by a capacitive sensor in which two electrodes form the “plates” of an electrical capacitor and in which these electrodes/plates are fixed in their spacing and orientation relative to one another and this is also permanently the case. The fixed positioning of the electrodes relative to each other changes the capacitance because either electrically conductive material or a dielectric is brought into the immediate vicinity of the electrodes. In the capacitive sensor according to the invention, therefore, there is no change in capacitance due to a change in the effective plate area, for example, by moving the plates relative to one another as in a variable capacitor, nor is there any change in capacitance due to a plate being moved or deformed by the effect to be measured, which would change the plate spacing and thus the electrically measurable capacitance. In contrast to the capacitive sensor according to the invention, the latter measurement principles require that the capacitive sensor be brought into contact with the measured object. Furthermore, the sensor may have only one electrode and the capacitance between this active electrode and the electrical ground potential is measured. Such sensors work with an oscillator whose frequency-determining capacitance is partly formed by the medium to be detected or by the environment. When the field of the probe capacitance is influenced by a conductor such as urine, the change in capacitance is due to a change in the effective permittivity in the area of the electrodes; the achievable switching distance is usually 60-80 mm. Capacitive proximity sensors usually have a calibration facility (potentiometer) to adapt the sensitivity or the switching thresholds to the conditions of use. Another circuit concept for a capacitive proximity sensor according to the invention works with three electrodes. In addition to the ground electrode and a measuring electrode, an additional excitation electrode is used. Advantages of this principle are a higher sensitivity with higher switching distances and a lower susceptibility to interference. It is also possible to detect media with very low permittivity or at a greater distance from the sensor.

According to the invention, the term “capacitive sensor” is thus synonymous with the term “capacitive proximity sensor”.

The toilet device according to the invention can be used to measure all physical parameters, i.e. all parameters directly or indirectly related to micturition. Preferably, the measured micturition parameters are selected from the group comprising: urine flow rate, micturition volume, micturition duration, micturition frequency, micturition speed, bladder pressure, pelvic floor pressure, urethral width and micturition characteristics such as intermittent urination or dribbling.

Since the capacitive sensor allows non-contact measurement of micturition parameters, it can be placed at a large number of different positions in or on the toilet device. Preferably, the capacitive sensor is mounted in the toilet device at one or more of the following positions:

on the housing inner wall,

on the outer wall of the housing,

inside the housing wall,

at the housing opening,

Here, it is particularly preferred that the capacitive sensor is mounted on the housing outer wall. Based on a common sitting toilet, this means an attachment to the outside of the toilet bowl.

In an alternative embodiment, the capacitive sensor is attached to the inner wall of the housing. This positioning allows a particularly sensitive measurement due to its proximity to the urine stream. In this embodiment, it is advantageous if the capacitive sensor is covered with a protective layer that is impermeable to water and resistant to urine. Such a protective layer expediently has a hydrophobic surface, which is preferably a surface with a lotus effect. As a result, urine beads off the sensor and the unwetted sensor continues to allow accurate measurement. Such a hydrophobic or lotus-effect surface makes the capacitive sensor particularly suitable for use in water-free toilets and urinals.

In another embodiment, the capacitive sensor is mounted on the outer wall of the housing. This positioning ensures that the sensor does not come into contact with the excrement. The outside positioning also allows for easy attachment (e.g., as part of a retrofit to a common toilet facility), maintenance, repair, removal, or replacement of the sensor.

In another embodiment, the capacitive sensor is mounted inside the housing wall. This positioning is also characterized by a low distance from the urine stream and is therefore also suitable for sensitive measurement. In addition, the capacitive sensor is thus optimally protected from damage or contamination by being enclosed or placed in the housing wall. Through this, the smooth surface (usually consisting of ceramic) of the toilet device remains intact, so that the toilet device can still be easily and thoroughly cleaned.

According to the invention, the attachment inside the housing wall also comprises an attachment to an inner side of a housing wall, insofar as this housing wall constitutes one of the two walls of a double-walled housing wall. Preferably, the capacitive sensor is mounted on the inside of the housing wall facing the interior of the toilet. In the sanitary ceramics sector, there are numerous urinals or toilets with double-walled bowls.

Alternatively, the sensor can be incorporated into the wall during the manufacture of the housing so that it is completely surrounded by the wall material. This can be done, for example, in the case of a housing made of plastic material, by casting it into the plastic housing during production.

It is expedient that the capacitive sensor has a sensor thickness of between 1 nm and 20 mm. Capacitive sensors can be designed with a very small thickness so that they can be flexibly adapted to the curves of the housing and can be attached to the housing without folding or kinking. The sensor thickness in this case is preferably between 1 nm and 20 mm, particularly preferably between 50 nm and 10 mm, and especially preferably between 200 nm and 3 mm.

In a further embodiment, the capacitive sensor comprises a lower carrier layer, a middle layer comprising the electrode and the lead(s), and an upper mounting layer for connecting the sensor to the housing or the housing opening, the carrier layer and the mounting layer preferably consisting of an electrically insulating material.

The capacitive sensor may be embedded in an insulating carrier matrix, such as plastic or a solid gel. Here, it is preferred that the surface of the carrier matrix has at least partially an adhesive layer that enables attachment to the housing.

The capacitive sensor expediently comprises a connection means for the electronical and/or electrical connection of the sensor to additional electronic or electrical components. The connection means is preferably selected from the group comprising metallic push button, cable, adhesive connection and plug part of a connector.

The capacitive sensor expediently comprises a data transmission module for wired or wireless data transmission. In this way, the measured data, i.e. parameters relating to micturition, can be transmitted to a storage and/or evaluation unit.

In one embodiment, the capacitive sensor is designed as a foil, which allows simple and wrinkle-free attachment even with complex housing shapes.

In a further embodiment of the invention, the sensor is vapor-deposited directly onto the housing or burned into the housing material, thus forming an integral part of the housing wall.

According to the invention, it may also be provided that the capacitive sensor comprises electrodes of a thin metal foil deposited on an electrically insulating layer. Such an insulating layer preferably consists of rubber or another elastomer or of a thermoplastic material. Particularly preferred is the use of a polyimide film, which has high resistance to chemicals as well as high temperature resistance and good insulating properties. Numerous polyimide films are known on the market, such as KAPTON from DuPont.

In order to be able to detect even small changes better, it is advantageous if, in the capacitive sensor according to the invention, the actual measuring electrode is surrounded by a shield electrode which shields the inhomogeneous edge region of the electric field from the measuring electrode. This results in an approximately parallel electric field between the measuring electrode and the normally grounded counter electrode with the known characteristic of an ideal plate capacitor.

In a further embodiment, the capacitive sensor has a shield that supports interference-free measurement of the micturition parameters in the housing.

In one embodiment, this may be a shield formed as a physical barrier, which is attached to the outside of the sensor, i.e., to the side of the toilet device facing away from the housing.

This shield is preferably an electronic shield. For this purpose, a shielding electrode is expediently used.

In one embodiment, the shielding electrode is mounted parallel to the measuring electrode and is controlled by a separate line.

The shielding electrode serves as protection from external interfering signals, but can also be used to improve measurement accuracy, temporal resolution, sensitivity, selectivity and measurement direction.

According to one embodiment, the control signal for the shielding electrode can only be a direct connection to “earth” (ground potential), or according to an alternative embodiment, it can also receive adapted voltage signals such as, for example, the inverted control signal of the measuring electrode or alternating signals.

Other control methods for the shielding electrode are also conceivable and depend on the intended use. If necessary, an additional amplifier circuit is required. In a further embodiment of the invention, the toilet device is characterized in that several capacitive sensors are arranged in the toilet device in such a way that they allow a two-dimensional or three-dimensional measurement of micturition. Here, an arrangement in or on the housing as a 2D array or 3D array is preferred.

In accordance with the invention, the capacitive sensor may be fixedly, conditionally releasably or reversibly releasably connected to the housing. Here, a reversibly releasable connection is preferred, insofar as the sensor can thus be easily replaced or transferred to another toilet device. Numerous connections are available to the skilled person for connecting the sensor to the housing. The following are mentioned here as examples: adhesive connection, adhesive-free adhesion connection, magnetic connection, Velcro fastener, zipper, screw connection, clamp connection, plug connection, snap connection and belt connection. Preferred here is the adhesive connection or the adhesive-free adhesion connection.

In a further embodiment of the invention, the toilet device is characterized in that the capacitive sensor allows the measurement of parameters present outside the housing of the toilet device, such as information on the localization of the user of the toilet device and/or the temporal and spatial variation of the user localization.

In a preferred embodiment of the invention, the toilet device is characterized in that it comprises a storage and/or evaluation unit (6) for storing and/or evaluating the measurement data.

In one embodiment of the invention, the toilet device may additionally comprise a temperature sensor and/or an acceleration sensor. It is preferred for this purpose that the temperature sensor permits non-contact measurement of the temperature of the urine. It is further preferred that the acceleration sensor allows non-contact measurement of the user's movement data, vibrations and structure-borne sound during urination.

The urine temperature can be measured directly or indirectly, e.g. directly by measurement of the urine stream or of the urine film in the housing, or by measuring urine splashes, urine spray or the air temperature in the direct urine environment.

When measuring the indirect urine temperature, it is preferable to perform a time-dependent evaluation of the measurement, since the urine usually cools down when it comes in contact with the housing of the toilet device and a useful urine temperature can only be recorded after a few seconds, after the housing has warmed up accordingly.

Furthermore, it is preferred that during the measurement of the urine temperature further temperature data, such as the temperature of the housing wall (or parts thereof) or the air temperature, are recorded and used for the evaluation of the urine temperature.

In this context, it is also intended according to the invention to record the length of the urine stream from exit from the body to the temperature measuring point, since the urine emerging from the body cools down due to the usually colder ambient air and the cooling increases accordingly with the length of the urine stream. A user profile for urination behavior, created by the toilet device, can also be used for this purpose.

In a preferred embodiment, the temperature sensor for non-contact measurement is designed as an infrared sensor. For this purpose, it has an infrared source that irradiates one or more areas within the housing of the toilet equipment and thus heats them to a target temperature T_{ziel}. The change in temperature in this/these area(s) resulting from the micturition is detected by the temperature sensor.

The temperature sensor is preferably attached to the housing interior or the housing opening of the toilet device.

In a preferred embodiment, since the temperature sensor and/or the acceleration sensor allow non-contact measurement of the micturition parameters, they can be placed at all possible positions in or on the toilet device. Preferably, the temperature sensor and/or the acceleration sensor are mounted in the toilet device at one or more of the following positions:

on the housing inner wall,

on the outer wall of the housing,

inside the housing wall,

at the housing opening.

Here, it is particularly preferred that the temperature sensor and/or the acceleration sensor are mounted on the housing outer wall. Based on a common sitting toilet, this means an attachment to the outside of the toilet bowl.

In an alternative embodiment, the temperature sensor and/or the acceleration sensor are mounted on the inner wall of the housing. This positioning allows a particularly sensitive measurement due to its proximity to the urine stream. In this embodiment, the sensor should be covered with a protective layer that is impermeable to water and resistant to urine. Such a protective layer expediently has a hydrophobic surface, which is preferably a surface with a lotus effect. In this way, urine beads off the sensor and the unwetted sensor continues to allow precise measurement. Such a hydrophobic surface or a surface with a lotus effect makes the temperature sensor and/or the acceleration sensor particularly suitable for use in water-free toilets and urinals.

In a further embodiment, the temperature sensor and/or the acceleration sensor are mounted on the outer wall of the housing. This positioning ensures that the sensor does not come in contact with the excrement. The outside positioning also allows for easy attachment (e.g., as part of a retrofit to a common restroom facility), maintenance, repair, removal, or replacement of the sensor.

In another embodiment, the temperature sensor and/or the acceleration sensor are mounted inside the housing wall. This positioning is also characterized by a short distance from the urine stream and is therefore also suitable for sensitive measurement. In addition, the temperature sensor and/or the acceleration sensor are optimally protected from damage or contamination by being enclosed or inserted in the housing wall. Hereby, the smooth surface (usually consisting of ceramic) of the toilet device is maintained, so that the toilet can still be cleaned easily and thoroughly.

According to the invention, the attachment inside the housing wall also comprises an attachment to an inner side of a housing wall, insofar as this housing wall constitutes one of the two walls of a double-walled housing wall. Preferably, the temperature sensor and/or the acceleration sensor are attached to the inside of the housing wall facing the interior of the toilet. In the sanitary ceramics sector, there are numerous urinals or toilets with double-walled basins.

Alternatively, the temperature sensor and/or the acceleration sensor can be incorporated into the wall during the manufacture of the housing so that it is completely surrounded by the wall material. This can be done, for example, in the case of a housing made of plastic material, by casting it into the plastic housing during production.

Expediently, the temperature sensor and/or the acceleration sensor have a sensor thickness of between 1 nm and 20 mm. Capacitive sensors can be designed with a very small thickness so that they can be flexibly adapted to the curves of the housing and can be attached to the housing without wrinkling or buckling. The sensor thickness in this case is preferably between 50 nm and 10 mm and particularly preferably between 200 nm and 3 mm.

In a further embodiment, the temperature sensor and/or the acceleration sensor comprise a lower carrier layer, a middle layer having the measurement electronics and the lead(s), and an upper mounting layer for connecting the temperature sensor and/or the acceleration sensor to the housing or the housing opening, the carrier layer and the mounting layer preferably consisting of an electrically insulating material.

The temperature sensor and/or the acceleration sensor can be embedded in an insulating carrier matrix, such as plastic or a solid gel. Here it is preferred that the surface of the carrier matrix has at least partially an adhesive layer, which enables the attachment to the housing.

The temperature sensor and/or the acceleration sensor expediently comprise a connection means for electronically or electrically connecting the sensor to additional electronic or electrical components. The connection means is here preferably selected from the group comprising metallic push buttons, adhesive connection, cable and plug part of a connector.

The temperature sensor and/or the acceleration sensor expediently comprise a data transmission module for wired or wireless data transmission. Thus, the acquired measurement data can be transmitted as parameters relating to the micturition, to a storage and/or evaluation unit.

Further data of the sensors claimed herein can be sent cumulatively or alternatively to an evaluation unit, which can be located, for example, in the housing of the device and/or on the guide part of the device.

Furthermore, it is possible that cumulatively or alternatively data from the sensors and/or from the evaluation unit are transmitted to an external analysis unit. This could be, for example, a data processing and/or analysis application running on a smartphone or the like.

It is conceivable here that at least some of the sensors can be switched off or on depending on the urine to be analyzed, whereby the device can be operated more energetically efficiently.

It is understood that data or information obtained on site can also be transmitted to external devices, such as a data carrier, etc., for further evaluation or storage.

In one embodiment, the temperature sensor and/or the acceleration sensor are designed as a foil, which allows simple and flush surface mounting, i.e. without wrinkles or bubbles, even with complex housing shapes.

In a further embodiment of the invention, the temperature sensor and/or the acceleration sensor are vapor-deposited directly onto the housing or baked into the housing material, thus forming an integral part of the housing wall.

In a further embodiment of the invention, the toilet device is characterized in that a plurality of temperature sensors and/or acceleration sensors are arranged in the toilet device in such a way that they allow a two-dimensional or three-dimensional measurement of the temperature and/or acceleration. Here, an arrangement in or on the housing as a 2D array or 3D array is preferred.

According to the invention, the temperature sensor and/or the acceleration sensor can be fixedly, conditionally detachably or reversibly detachably connected to the housing. Here, a reversibly detachable connection is preferred, insofar as the temperature sensor and/or the acceleration sensor can thus be easily replaced or transferred to another toilet device. Numerous connections are available to the skilled person for connecting the sensor to the housing. Mentioned here by way of example are: adhesive connection, adhesive-free adhesion connection, magnetic connection, hook-and-loop fastener, zipper fastener, screw connection, clamp connection, plug connection, snap connection and belt connection. Preferred here is the adhesive connection or the adhesive-free adhesion connection.

In a further embodiment of the invention, the toilet device is characterized in that the temperature sensor, which is preferably arranged for contactless measurement of the urine temperature, further preferably has one or more of the following properties:

The temperature sensor is arranged to allow measurement of the stream temperature during urination;

The temperature sensor is arranged to allow measurement of the surface temperature of the inside of the housing and its variation over time;

The temperature sensor is an infrared sensor;

The temperature sensor is a 1 D, 2D or 3D sensor.

In a second aspect, the invention provides a method for measuring micturition parameters in a toilet device, the toilet device comprising a housing having a housing opening and a capacitive proximity sensor, the method comprising the steps of: (a) non-contact measurement of micturition parameters during urination by the capacitive proximity sensor;

b) optional measurement of the urine temperature during urination by means of a temperature sensor additionally incorporated in the toilet device;
c) optional measurement of user movement data, vibrations and structure-borne sound during urination via an acceleration sensor additionally installed in the toilet device;
d) wired or wireless transmission of the measurement data collected in steps a) to c) to an evaluation unit; and
e) evaluation of the measurement data in the evaluation unit.

In a preferred embodiment of the invention, the procedure described above is carried out with the toilet device according to the invention.

In a further embodiment of the method according to the invention, the evaluation of the measured data in the evaluation unit is carried out on the basis of a model or via calibration curves. The model-based evaluation is preferably based on a mathematical, physical, physiological or medical model, or it is carried out using pattern recognition algorithms.

In one embodiment of the invention, the toilet system additionally has a toilet flush whose characteristics, such as flush water quantity and/or flush water duration and/or flush water temperature, are used as reference values for the evaluation.

In a third aspect, the invention relates to the use of a capacitive sensor for measuring micturition parameters in a toilet device. Preferably, in this aspect, the sensor is used for measuring micturition parameters in the toilet device according to the invention. Also preferably, in this use, the measurement of the micturition parameters is carried out according to the method according to the invention described above.

It is understood that the features of the solutions described above or in the claims can also be combined, if necessary, in order to be able to implement the advantages and effects achievable in a correspondingly cumulative manner.

At this point it is to be mentioned that in the context of the present patent application the expression “in particular” is always to be understood as indicating an optional, preferred feature. The expression is not to be understood as “and indeed” and not as “namely”.

Furthermore, it should be noted that in the context of the present patent application, indefinite articles and indefinite numerals such as “one . . . ”, “two . . . ” etc. are generally to be understood as indicating a minimum, i.e. as “at least one . . . ”, “at least two . . . ” etc., unless it is clear from the context or the specific text of a particular passage, for example, that only “exactly one . . . ”, “exactly two . . . ” etc. are intended.

In addition, further features, effects and advantages of the present invention are explained with reference to the accompanying drawing and the following specification, in which a device for on-site analysis of excrements is illustrated and described by way of example.

Components which in the individual figures at least substantially correspond with respect to their function, can be marked here with the same reference numbers, where the components do not have to be numbered and explained in all figures.

It should be said here that the figures shown are representations illustrating the principal structure and the principal mode of operation.

In the drawings:

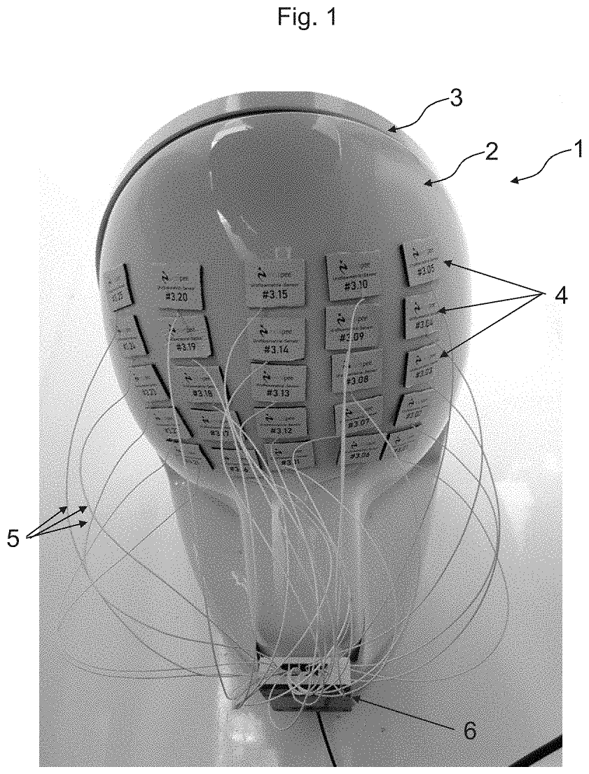

FIG. 1 shows schematically a first perspective view of a partially shown toilet device according to an embodiment of the invention with a 2D array of capacitive sensors;

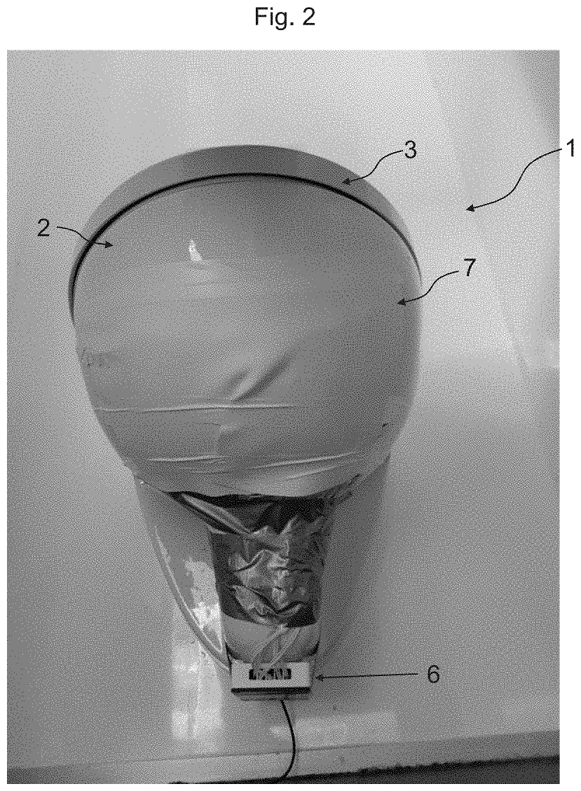

FIG. 2 shows schematically a perspective view of a partially depicted toilet device according to a second embodiment of the invention with a cover of the capacitive sensors mounted on the outside;

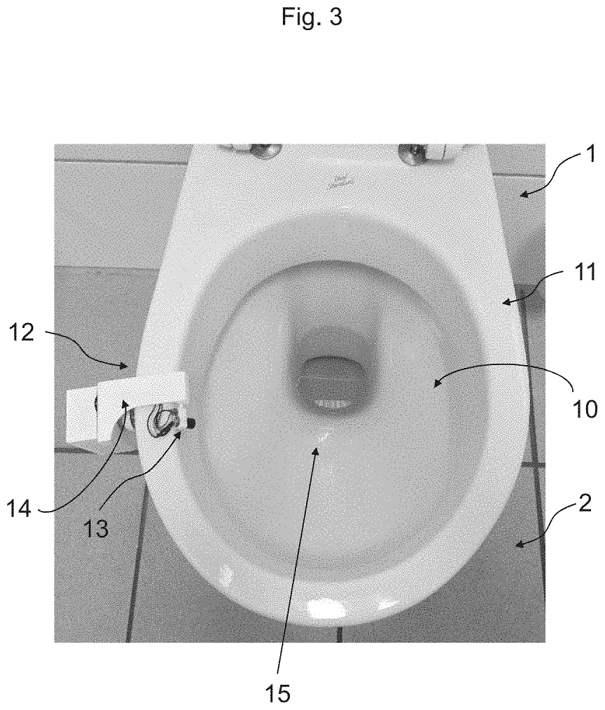

FIG. 3 shows a schematic perspective view of a partially depicted toilet device according to a further embodiment of the invention with a temperature sensor.

FIG. 1 shows a toilet device 1 with a toilet bowl 2 and a toilet seat 3. The capacitive sensors 4 are glued to the outer wall of the toilet bowl as a 2D array of 5×5 sensors in an almost square arrangement and are each connected by a cable 5 to the power source with evaluation and data transmission unit 6.

FIG. 2 shows the toilet device 1 according to FIG. 1 with a toilet bowl 2 and a toilet seat 3. The capacitive sensors 4 connected to the power source with evaluation and data transmission unit 6 are completely and waterproof covered here by a cover 7.

FIG. 3 shows a toilet device 1 with a toilet bowl 2 and its opening 10. By folding up the toilet seat 3 (not shown here), which rests on the edge of the bowl 11, the temperature sensor 12, which is fastened to the wall of the toilet bowl by means of the holding device 14 designed as a clamping device, becomes completely visible. The temperature sensor 12 has a head 13 that includes an infrared sensor, which is oriented towards the measuring point 15 and allows the temperature to be measured there.

At this point, it should be explicitly pointed out that features of the solutions described above or in the claims and/or figures can also be combined, if necessary, in order to be able to implement or achieve the explained features, effects and advantages in a correspondingly cumulative manner.

It is understood that the exemplary embodiment explained above is merely a first embodiment of the device according to the invention. Therefore, the embodiment of the invention is not limited to this embodiment.

All features disclosed in the application documents are claimed to be essential to the invention insofar as they are new, individually or in combination with each other, compared to the prior art.

### LIST OF REFERENCE NUMBERS USED

1 toilet device
2 housing (toilet bowl)
3 toilet seat
4 capacitive proximity sensors
5 cable
6 power source with evaluation and data transmission unit
7 sensor cover
10 housing opening
11 housing edge (edge of toilet bowl)
12 temperature sensor
13 head of temperature sensor
14 holding device for temperature sensor
15 temperature measuring point

1. Toilet device for measuring micturition parameters, comprising:
a housing having a housing opening for receiving urine;
a capacitive sensor for time-dependent measurement of micturition parameters, wherein the capacitive sensor is a capacitive proximity sensor.

2. Toilet device according to claim 1, wherein the toilet device is selected from the group comprising toilet, urinal, bidet, commode chair, squat toilet and chamber pot.

3. Toilet device according to claim 1, wherein the measured micturition parameters are selected from the group comprising urine flow rate, micturition volume, micturition duration, micturition frequency, micturition velocity, bladder pressure, pelvic floor pressure, urethral width and micturition characteristics such as intermittent urination or dribbling.

4. Toilet device according to claim 1, wherein the capacitive sensor is mounted in the toilet device at one or more of the following positions:
at the housing inner wall,
at the housing outer wall,
inside the housing wall,
at the housing opening,
wherein attachment to the housing outer wall is preferred.

5. Toilet device according to claim 1, wherein capacitive sensor has one or more of the following properties:
sensor thickness between 1 nm and 20 mm,
the sensor comprises a lower carrier layer, a middle layer comprising the electrode and the lead, and an upper mounting layer for connecting the sensor to the housing or the housing opening, the carrier layer and the mounting layer preferably consisting of an electrically insulating material;
the sensor is mounted in an insulating support matrix, such as plastic or a solid gel, the surface of the support matrix having at least partially an adhesive layer;
the sensor comprises a connection means for electronic or electrical connection, preferably selected from the group comprising metallic push buttons, adhesive connection, cable and plug part of a connector;
the sensor comprises a data transmission module for wired or wireless data transmission;
the sensor is designed as a foil;
the sensor is directly vapor-deposited on the housing or burned into the housing material;
the sensor comprises electrodes made of a thin metal foil deposited on an electrically insulating layer made of, for example, rubber or a flexible plastic such as polyimide foil.

6. Toilet device according to claim 1, wherein several capacitive sensors are mounted in the toilet device in such a way that they allow a two-dimensional or three-dimensional measurement of micturition, and in this case are preferably arranged in or on the housing as a 2D array or 3D array.

7. Toilet device according to claim 1, wherein the capacitive sensor is connected to the housing in a fixed, conditionally detachable or reversibly detachable manner, a reversibly detachable connection being preferred and reversibly detachable connection being particularly preferably selected from the group comprising: suction cup, adhesive connection, adhesive-free adhesion connection, magnetic connection, Velcro fastener, zipper fastener, screw connection, clamp connection, plug connection, snap connection and belt connection.

8. Toilet device according to claim 1, wherein the capacitive sensor allows the measurement of parameters present outside the housing, such as information on the localization of the user of the toilet device and their temporal and spatial variation.

9. Toilet device according to claim 1, wherein it additionally comprises a temperature sensor and/or an acceleration sensor.

10. Toilet device according to claim 1, wherein the temperature sensor is set up for contactless measurement of urine temperature, and preferably has one or more of the following properties:
the temperature sensor is arranged to allow measurement of the urine temperature during urination;
the temperature sensor is adapted to allow measurement of the surface temperature of the inside of the housing and its variation over time;
the temperature sensor is an infrared sensor;
the temperature sensor is a 1D, 2D or 3D sensor.

11. Method for measuring micturition parameters in a toilet device comprising a housing having a housing opening and a capacitive proximity sensor, the method comprising the following steps:
(a) non-contact measurement of micturition parameters during urination by the capacitive proximity sensor;
b) optional measurement of the urine temperature during urination by means of an additional temperature sensor incorporated in the toilet device;
c) optional measurement of the user's movement data, vibrations and structure-borne sound during urination via an acceleration sensor additionally incorporated in the toilet device;
d) wired or wireless transmission of the measurement data collected in steps a) to c) to an evaluation unit;
e) evaluation of the measurement data in the evaluation unit.

12. Method according to claim 11, wherein the method is carried out with a toilet device.

13. Method according to claim 11, wherein the evaluation of the measurement data in the evaluation unit is carried out model-based or via calibration curves, the model-based evaluation preferably being based on a mathematical, physical, physiological or medical model or running via pattern recognition algorithms.

14. Method according to claim 1, wherein the toilet device additionally has a toilet flush, the characteristics of which, such as flush water quantity and/or flush water duration and/or flush water temperature, are used as reference values for the evaluation.

15. Use of a capacitive proximity sensor for measuring micturition parameters in a toilet device.
